(19) United States
(12) Patent Application Publication
(10) Pub. No.: US 2009/0081926 A1
(43) Pub. Date: Mar. 26, 2009

(54) Abdominal support garment with post-surgical incision protection

(76) Inventor: Melinda Gros, New York, NY (US)

Correspondence Address: Peter S. Canelias;Law Offices of Peter S. Canelias, Suite 2148, 420 Lexington Avenue, New York, NY, 10170, US

(21) Appl. No.: 11/903,151

(22) Filed: Sep. 20, 2007

(51) Int. Cl. A41C 1/08; A41B 9/04; A61F 5/24

(52) U.S. Cl. 450/155; 128/96.1; 2/406

(57) ABSTRACT

This invention relates generally to a therapeutic medical garment that supports the abdomen or belly of a pregnant or post-partum woman and of either men or women with distended abdomens. The invention relates to an adjustable-support therapeutic undergarment which lifts the abdomen to prevent irritation of a surgical incision or scar in the lower abdominal or groin region such an incision resulting from a caesarian section or a hernia incision. The invention allows proper healing of the incision, and relieves both stress and strain on the scar and friction from the overhanging abdomen.

### BACKGROUND OF THE INVENTION

This invention relates generally to a therapeutic medical garment, specifically a support undergarment to be worn by pregnant and post-partum women and by either men or women with distended or sagging abdomens. More particularly, the invention relates to an adjustable-support therapeutic undergarment which lifts the abdomen to relieve strain and prevent frictional irritation of a surgical incision or scar in the lower abdominal or groin region, such as a scar resulting from a prior caesarian section or from hernia surgery.

When a patient has had a surgery with an incision in the lower part of the abdomen or groin, it is important for recovery that the site not be subjected to rubbing, strain, pulling or other disturbances so that the incision may properly heal, and after healing, not reopen. For women in particular, however, a subsequent pregnancy with the additional weight and size of the fetus creates considerable strain and discomfort on the ligaments of the abdominal muscles in the pregnant woman and may weaken the incision from a prior caesarian delivery.

Previous attempts to protect surgical incisions in the abdominal area have structures that compress the abdominal area so as to aid healing. For example, U.S. Pat. No. 6,238,124 to Schleuning et al. discloses a form fitting garment and one or more compression pads disposed in pouches located on the interior of the garment. These previous attempts do not address the problem of abdominal overlap of the incision site.

There is also a need for a combination of a support structure which supports a large abdomen or belly and which is adjustable to provide support for the full range of sizes and shapes which occur during pregnancy or weight loss or gain. For such a garment to be worn for long periods of time, it must be lightweight and comfortable, balancing ergonomic support with flexibility. The garment is designed to provide enough rigid support to adequately protect the surgical incision without overly restricting abdominal movement. And, such a garment should be designed to allow for an attractive appearance.

### BRIEF SUMMARY OF THE INVENTION

The invention is a medical (therapeutic) garment for use as a support undergarment by persons with post-surgical groin or abdominal incisions in cases where the subject's belly and abdomen put pressure on the incision. The invention cushions the incision site and supports the belly and abdomen away from the incision site to preserve the integrity of the incision and to prevent undue strain and frictional irritation of the incision. Lifting the belly or abdomen also prevents other deleterious effects on the incision such as the trapping of moisture under the folds of the skin which can harm the integrity of the sutures as well as causing surface irritation and possible infection, bacterial, viral or fungal. The garment can be worn under clothing and can replace other undergarments covering the belly, abdomen and groin so that the garment can be worn continuously.

The garment is comprised of a panty with an overlayer, a preferably removable and adjustable support belt with elastic properties, and a crotch piece. The front of the garment has a stabilizer cushion that is preferably oriented in the direction of the incision, i.e., a substantially vertical stabilizer cushion for substantially vertical incisions and a substantially horizontal stabilizer cushion for substantially horizontal incisions. The back section preferably comprises channeling for a removable, under-the-belly elastic support belt.

When assembled, the garment is preferably comprised of at least two layers stitched or sewn together; a first layer against the user's body providing a covering for the user's abdomen and comprises the panty and crotch and a second layer that provides lateral support and wraps around the user. Alternatively, a third layer between the first layer and the second layer that wraps around the user, may provide for additional support.

The first layer is preferably formed from a stretchable fabric for covering the abdomen so that it can adjust to increases in girth during pregnancy or to weight gain or weight loss. The elastic support belt preferably has a band width of about 3 inches, the precise width correlated with the weight and size of the user. If correlation of the band width with the weight and size of the user suggests that a wider support belt is necessary or desirable, two bands are preferably used as opposed to a single elastic band of 5 inches or more, due to the propensity of the wider bands to double over and to distribute the load or tension to the peripheral edges along the width of the band instead of throughout the width of the band. This too-wide elastic can cause the wider band to cut into the user along the edges. The band includes differential support structure to provide more support for the sides and back of the user as compared with the front of the person about which the band has been wrapped.

The elastic belt is preferably adjustable and secured with hook and loop fasteners such as Velcro®. The fasteners are preferably located in the front of the garment to allow the user to easily adjust the tension of the strap without having to reach around to the rear portion of the garment. Due to the objective of the invention, it is important to locate the elastic band beneath the protruding abdomen or belly so as to lift the protrusion off of and away from the incision site, and help prevent creasing or folding of the skin about the incision site. The crotch area has a stabilizer cushion on the interior side of the garment to provide cushioning and stabilization to the incision site. The stabilizer cushion is preferably formed from fleece or cushioned layers. It may also include a moisture-resistant backing on the non-body-contacting side away from the user's body to prevent any oozing or seepage from the incision from migrating to the front of the garment and possibly staining the user's outer garments. The stabilizer cushion may also be treated with an anti-microbial preparation to inhibit the formation of infection at the site and may also have wicking properties or other moisture management properties to draw bodily fluid away from the wound site. Another embodiment may employ a gel layer or layers in conjunction with the stabilizer cushion to provide additional comfort and support for the user.

In other embodiments, the stabilizer cushion may be affected by employing a pad of the disposable type used in wound dressings. It has been generally known in the art to provide disposable absorbent pads. For example, U.S. Pat. No. 4,578,066 to O'Conner (incorporated by reference herein for all purposes) shows a type of such pad comprised of a pervious fabric, an absorbent material, and an impervious liner. The absorbent material forming the inner portion of the pad is connected to a pervious body-side liner material such as a fabric with wicking properties. The connection of pervious liner to absorbent is such that when the pad is manipulated the pervious liner material stays in contact with the absorbent material and aids in transfer of liquid through the pervious liner into the absorbent. The fabric forming part of the impervious layer is preferably hydrophobic.

The layers are preferably stitched or sewn together into a unitary garment. The garment may also be formed by a seamless knitting process, where the fabric is not cut and sewn but rather is knit or woven continuously with graduated compression within the fabric substituting for stitched layers of fabric. In other embodiments, the garment may be made by a felting or similar method where the garment is not cut and sewn but rather is molded with graduated compression part of the process.

The object of the invention is to provide a garment which both protects and stabilizes groin and abdominal incisions and attendant scars and lifts and maintains the abdomen away from the incision site.

Another object of the invention is to protect, cushion and stabilize groin and abdominal incisions and attendant scars and lift the abdomen away from the incision site, a single, unitary garment accomplishing these therapeutic objects.

Another object of the invention is to provide an incision support area that flexes in the direction of the surgical incision to reduce strain on the scar tissue.

Other objects and advantages will be more fully apparent from the following disclosure and appended claims.

### BRIEF DESCRIPTION OF THE DRAWINGS

The above-mentioned and other features and objects of this invention and the manner of obtaining them will become apparent and the invention itself will be best understood by reference to the following description of an embodiment of the invention taken in conjunction with the accompanying drawings, wherein:

FIG. 1 is a right-front perspective view of a preferred embodiment of the invention being worn by a pregnant woman;

FIG. 2a is a front view of the top layer of the garment;

FIG. 2b is a rear view of the top layer of the garment;

FIG. 3a is a front view of an elastic belt for use with the garment with the preferred hook and loop fastener in the secured position;

FIG. 3b is a rear view of an elastic belt for use with the garment;

FIG. 4a is a front view of the middle layer of the garment;

FIG. 4b is a rear view of the middle layer of the garment;

FIG. 5a is a front view showing the panty portion of the garment;

FIG. 5b is a rear view showing the panty portion of the garment;

FIG. 6 is a front view outlining the cushioning layer with the crotch piece positioned over the incision site;

FIG. 7 is a side view of a second embodiment of the invention;

FIG. 8 is a side view of a third embodiment of the invention;

FIG. 9 is a side view of a fourth embodiment of the invention having two belts;

FIG. 10 is a side view of a fifth embodiment of the invention having two belts.

### DETAILED DESCRIPTION

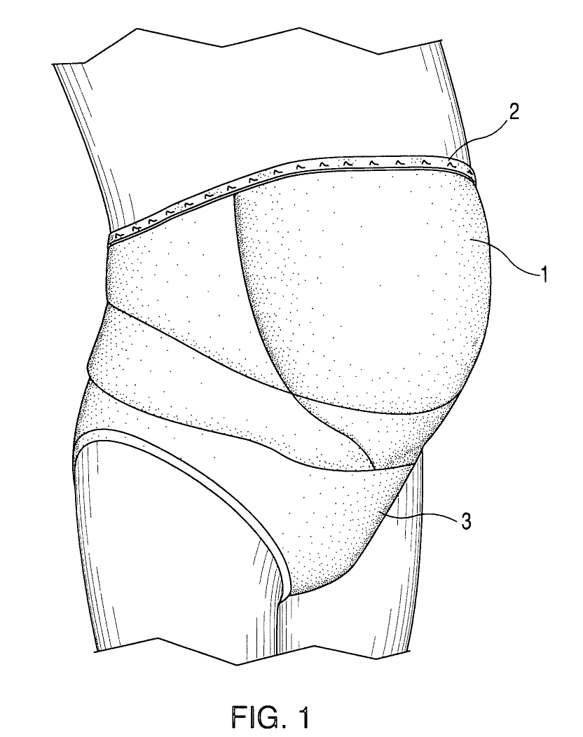

A preferred embodiment of this invention is shown in FIG. 1, here an approximately medium size being worn by a pregnant woman. The first layer or overlayer is formed to be against the body of the user in the upper abdomen area, providing support for the upper abdomen. The support panel 1 comprises the exposed upper portion of the first layer covering a substantial portion of the protruding abdomen. In other embodiments, the first layer may be modified to cover more or less of the protruding abdomen. Here, the support panel 1 is bounded by an upper seam 2 that extends over and around the top portion of the protruding abdomen. The fabric of the upper seam 2 is preferably folded over to help keep the garment in place and to contain the elastic material. The upper seam is preferably formed of an elastic material similar to those used in conventional undergarments. Thus the top edge of the exposed upper portion of the first layer provides additional anchoring for the garment to prevent slippage and keep the other functional components in proper alignment with the user's body.

The support panel 1 is preferably made of a material that is biaxially stretchable, with greater relative elasticity in the axial direction extending from the top edge to the crotch edge of the panel. The support panel 1 is shown here as being the panty 3, but is positioned to provide the proper stretch. The panty 3 provides stretch in its width to comfortably receive the expansion of the hips and waist of the pregnant woman or overweight person. Layering support panel 1 provides firm ergonomic support to the abdominal area while sacrificing only a minimal amount of relative elasticity in the direction extending from side-to-side.

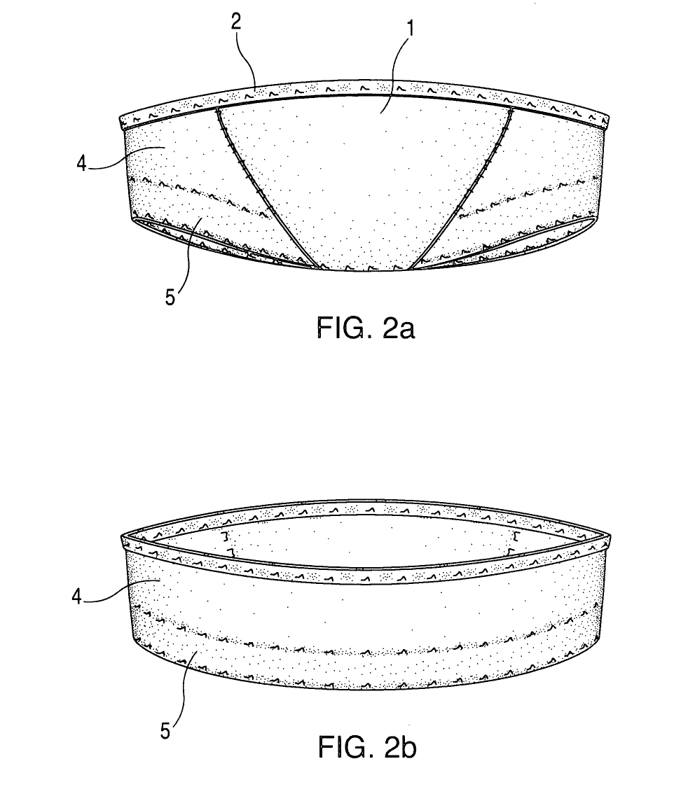

FIG. 2a shows the overlayer with support panel 1 having a stitched lateral support 4. The lower portion of lateral support 4 has a stitched channel 5 stitched through the support panel fabric and the panty fabric to contain an elastic belt encircling the lower abdomen of the user. FIG. 2b shows the rear of the lateral support 4 and the continuation of the stitched channel 5 about the back of the user. FIGS. 2a and 2b show a preferred construction that places the channel 5 at the lower part of the opening for the belly.

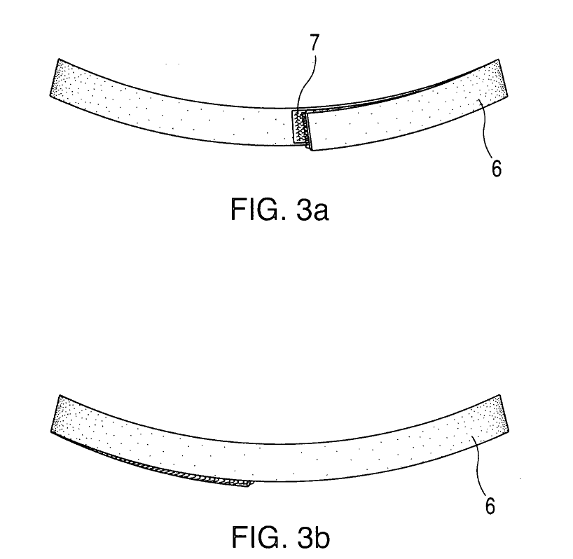

Turning now to FIG. 3, a front view of the elastic band 6 for encircling the body of the user through the channel 5 is shown. FIG. 3b shows the back of the elastic band 6. The elastic band 6 is preferably formed of an elastic material, and terminates in mating ends each fitted with a corresponding patch of material having hook and loop fasteners such as Velcro® so that the elastic band 6 is secured at closure 7 in the front of the user's body for easy access, securing and detaching and adjustment. This is important because the purpose of the belt is to hold away the abdomen or belly from the incision site, rather than merely supporting the abdomen. The elastic band 6 is preferably about 3 inches in width, positioned proximate the incision site and above the crotch portion. Alternatively, two elastic bands may be employed so as to avoid an overly wide elastic (five inches or greater), which would transfer stress to the edges of the band and cause discomfort for the user. The two elastic bands would be preferably disposed one above the other encircling the lower abdomen of the user, and may use separate channels (not shown) for independent adjustability of each band.

Continuing with FIG. 3, the fasteners are preferably made of corresponding patches of material different in length from one another, such that the loop patch is longer than the patch comprising the hooks, so that the elastic band 6 is adjustable over the length of the loop patch. The loop patch is preferably about three inches in length and extends the full width of the elastic band 6. The hook patch is preferably about one inch in length and extends the full width of the elastic band 6. Both patches are preferably stitched to the respective ends of the elastic band, with the loop patch on the upper exposed surface of one end and the hook patch on the lower surface of the second end, such that when in the secured position 7 the hook and loop fasteners are in mating engagement to securely hold the ends of the elastic belt in the secured position.

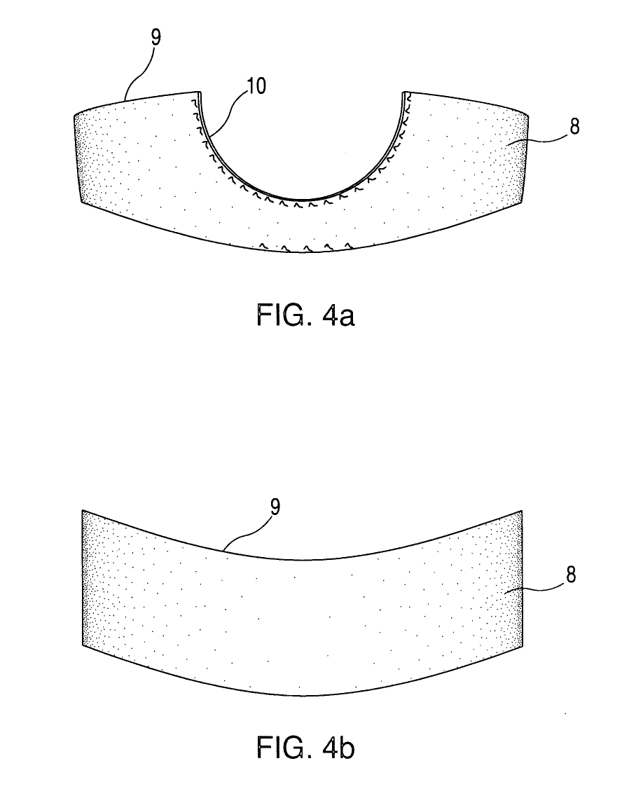

FIG. 4a shows a front view of the middle layer 8 of the garment, which is preferably stitched to the panty layer. Preferably, the top edge 9 of the middle layer preferably has a curved portion 10 to conform to the middle to lower part of the abdomen to avoid unnecessary restriction or compression, and the top edge 9 of the middle layer, when in use, arches upwards and rearwards about the body of the user. FIG. 4b shows the rear or back portion of the middle layer 8. This layer, however, may or may not circle around the body to include the back area of the user and can terminate without encircling the back of the body of the user. This configuration of the middle layer provides a form of sling for the abdominal region without causing any undue compression or discomfort. In other embodiments, the middle layer may be modified according to the relative size of the user and the need to provide additional Support to the user.

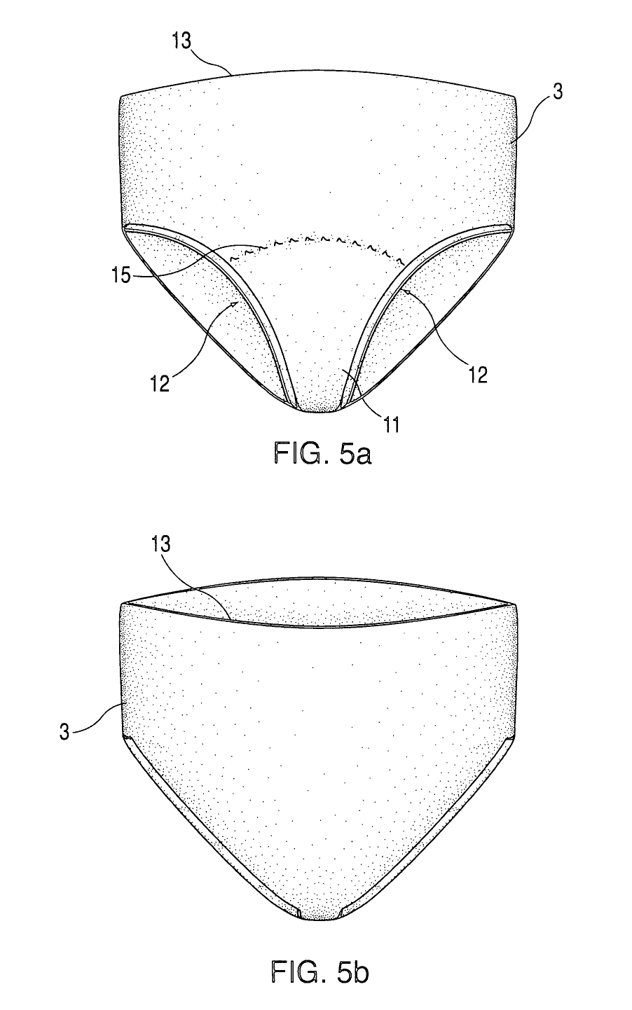

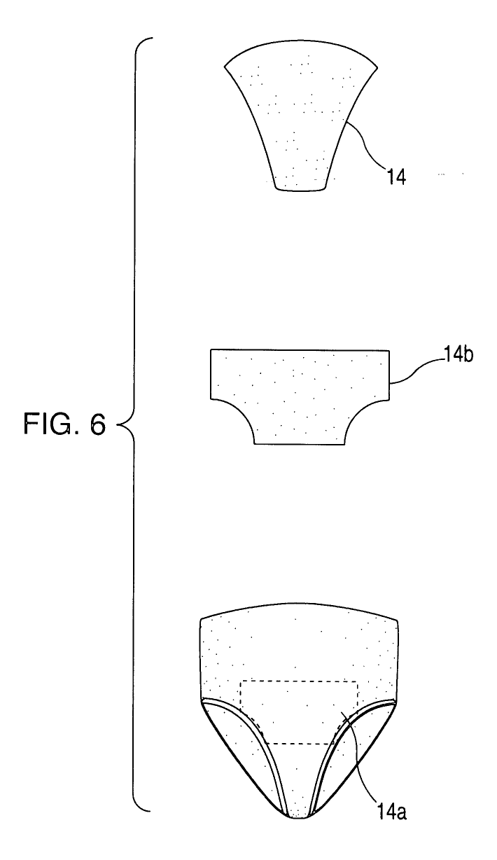

FIG. 5a shows the front of the panty portion 3 of the garment, comprising a crotch portion 11 with leg cutouts 12 for typical use. Where necessary, stitching may be used to hold the cushioning unit in place along the leg cutouts. In other embodiments, where the cushioning unit may not extend from the leg cutouts, stitching may not be necessary. When stitched, the edge 12 of the crotch portion 11 is designed to help keep the panty in a fixed position. FIG. 5b shows the rear of the panty portion 3. The upper edge 13 of the panty portion 3 is preferably stitched to the top portion of the overlayer, as shown in FIG. 2. Leg cutouts 12 have stitching about the edge preferably folded over to help keep a cushioning layer 14b (FIG. 6) in place against the user's body. The panty portion 3 holds the cushioning layer 14b in place preferably by means of stitching 15. The lower section of the panty may also include a panel of powernet or similar supportive material for stabilization and compression over the pubic symphysis to help relieve the stresses of pregnancy.

The panel of powernet is preferably composed of strong wide band of elastic material located below the adjustable elastic banding described in FIG. 3. The panel of powernet is preferably inserted between the layer of material comprising the panty and the layer of material comprising the crotch piece. Ideally, the powernet panel would stretch across the pubic symphysis from the right leg opening 12 to the left leg opening 12. The band may be widened to extend the support from the front lower abdomen to under the wearer's crotch or even up to the channeling for the adjustable elastic banding in the back. The power net panel could either be a single supportive piece or could be layered to provide firmer support. It could even encircle the lower abdomen, front and back and across the wearer's crotch. In a more baroque incarnation, the powernet may be shaped to extend further up the front leg openings 12, shown in FIG. 5a, even to the top of the garment, having the top of the powernet panel curve downward toward the center below the belly button to accommodate the shape of a pregnant belly. Depending on the size of the powernet panel, it may, but does not have to, overlap the stabilization pad.

FIG. 6 shows a possible location of the cushioning layer 14b, preferably an inner layer outlined just above the crotch portion 14 that is soft and comfortable. The cushioning layer 14b is shown separately, and is shown in situ as 14a. The cushioning layer may be made up of a single layer or a plurality of layers. The cushioning layer has a stabilizer cushion 141) on the interior side of the garment to provide cushioning and stabilization to the incision site. The stabilizer cushion may include a moisture-resistant backing on the non-body-contacting side away from the user's body to prevent any oozing or seepage from the incision from migrating to the front of the garment and possibly staining the user's outer garments. These layers may also be waterproof, although a waterproof layer is not required. The stabilizer cushion may also be treated with an anti-microbial preparation to inhibit the formation of infection at the site and may also have wicking properties to draw bodily fluid away from the wound site.

A separate, disposable pad may also be used either in place of or in conjunction with the stabilizer cushion 14b. It has been generally known in the art to provide disposable absorbent pads, such as that disclosed in U.S. Pat. No. 4,578,066 to O'Conner, which is incorporated by reference herein for all purposes. O'Conner discloses a pad comprised of a fluid-pervious fabric, an absorbent material, and a fluid-impervious liner. The absorbent material forming the inner portion of the pad is connected to a pervious body-side liner material. The connection of pervious liner to absorbent is such that when the pad is manipulated the pervious liner material stays in contact with the absorbent material and aids in transfer of liquid through the pervious liner into the absorbent. The fabric forming part of the impervious layer is preferably hydrophobic.

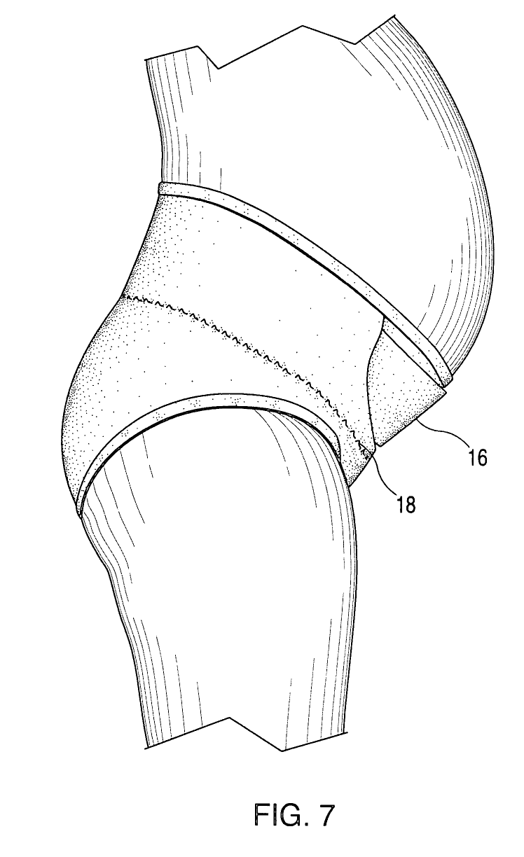

FIG. 7 shows side perspective of a second embodiment of the invention being worn by a pregnant woman. In this embodiment, the overlayer has a bottom edge and a top edge, both of which are stitched to the panty layer, creating a lateral support for the channel. The elastic band 16 passes through the channel created by the stitching of the overlayer and panty. As shown in FIG. 7, this embodiment does not extend the overlayer portion beyond the belly button, covering less of the abdomen than in the preferred embodiment of FIG. 1. In other embodiments, the overlayer portion may duplicate the panty portion, creating a version of the garment where the panty is made of a doubled fabric in which the top part may or may not have an opening for the abdomen. Where the panty is made of doubled fabric the cushioning and powernet support may be contained between the layers.

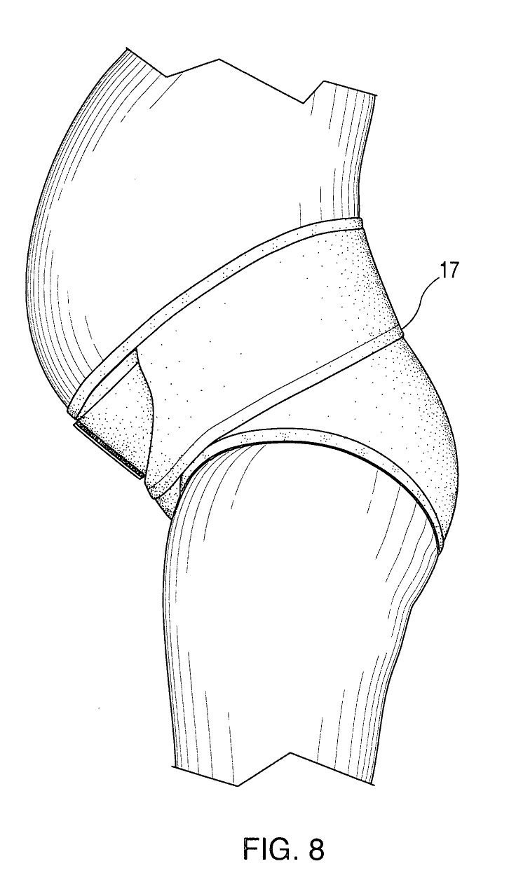

FIG. 8 shows side perspective of a third embodiment of the invention being worn by a pregnant woman. This third embodiment is the embodiment of FIG. 7 with an additional means for support. A second, middle seam 17, extends over and around the abdomen. Thus, the middle seam provides additional anchoring for the garment to prevent slippage and keep other functional components in proper alignment with the user's body.

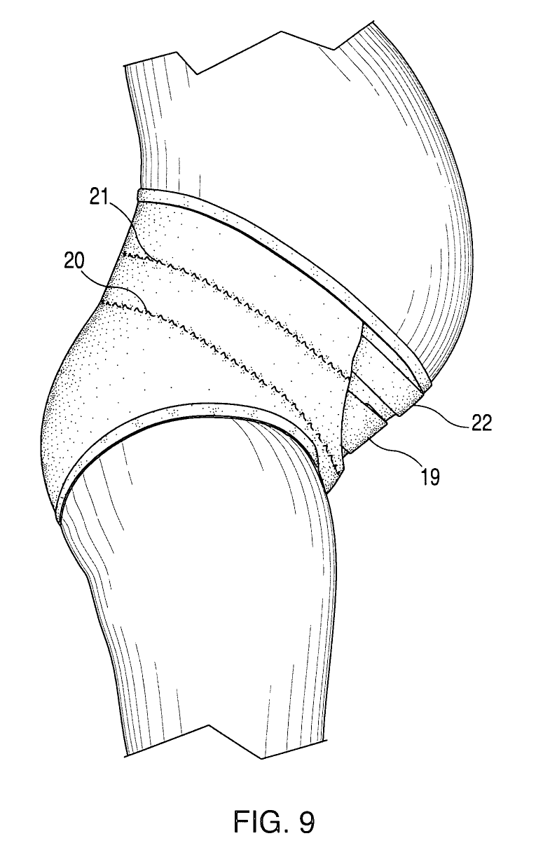

FIG. 9 shows side perspective of a fourth embodiment of the invention being worn by a pregnant woman. In this embodiment, the overlayer has a bottom edge and a top edge, both of which are stitched 20 to the panty layer for creating a lateral support for a first channel. The overlayer is further stitched 21 dividing the first channel, thereby creating a second channel. The two channels may contain two belts. The first belt 22 is the elastic belt described in FIG. 3. The second belt 19 may be a second elastic belt, but this is not required. In addition, the second belt 19 may have a different fastening means than the first belt 22 providing the user with a range of control with respect to the fit of the garment.

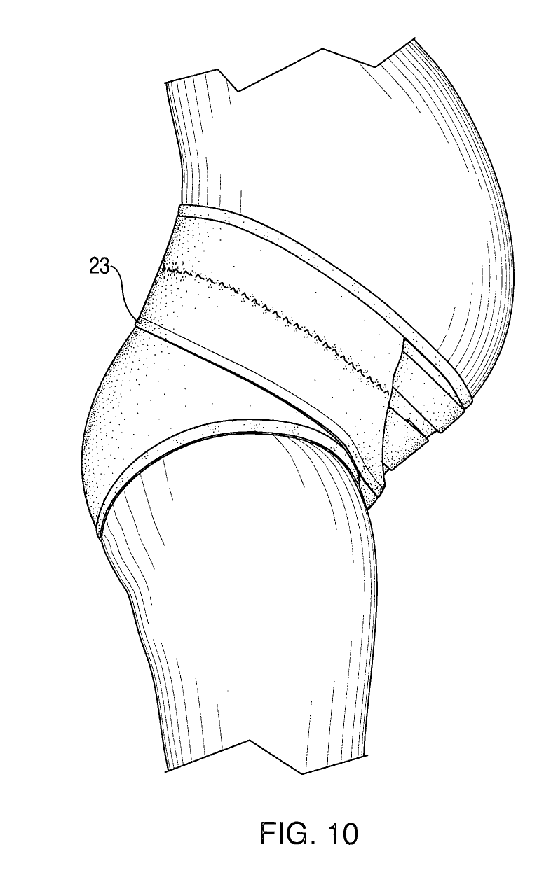

FIG. 10 shows side perspective of a fifth embodiment of the invention being worn by a pregnant woman. In this embodiment, the stitching 20 of FIG. 9 is replaced with a middle seam 23. Thus the middle seam provides additional anchoring for the garment to prevent slippage and keep other functional components in proper alignment with the user's body.

Since other modifications or changes will be apparent to those skilled in the art, there have been described above the principles of this invention in connection with specific apparatus, it is to be clearly understood that this description is made only by way of example and not as a limitation to the scope of the invention.

What is claimed is:

1. A therapeutic support garment comprising:
a top layer of fabric forming a support panel, the support panel having a stitched channel for supporting an adjustable belt;
the adjustable belt having a releasable fastener for fastening the adjustable belt around a lower section of a user's abdomen;
the stitched channel being above a bottom layer of fabric, the bottom layer of fabric forming a panty;
the panty having a body portion and a crotch portion; and
the crotch portion having a stabilizing cushion for protecting an incision.

2. The therapeutic support garment of claim 1, wherein the stitched channel is formed with a third layer of fabric.

3. The therapeutic support garment of claim 1, wherein the adjustable belt is approximately three inches in width.

4. The therapeutic support garment of claim 1, wherein the adjustable belt is a continuous band of elastic material with opposed overlapping ends thereof carrying the releasable fastener.

5. A therapeutic support garment comprising:
a top layer of fabric forming a support panel, the support panel having a first stitched channel and a second stitched channel;
the first stitched channel for supporting a first adjustable belt;
the second stitched channel for supporting a second adjustable belt;
the first adjustable belt having a releasable fastener for fastening the adjustable belt around a lower section of a user's abdomen;
the first stitched channel being above the second stitched channel; the second stitched channel being above a bottom layer of fabric, the bottom layer of fabric forming a panty;
the panty having a body portion and a crotch portion;
the crotch portion having a stabilizing cushion for protecting an incision.

6. The therapeutic support garment of claim 5, further comprising:
a third layer of fabric forming the first stitched channel; and
forming the second stitched channel.

7. The therapeutic support garment of claim 5, wherein the first adjustable belt and the second adjustable are approximately three inches in width.

8. The therapeutic support garment of claim 5, wherein the first adjustable belt is a continuous band of elastic material with opposed overlapping ends thereof carrying the releasable fastener.

9. The therapeutic support garment of claim 5, wherein the second adjustable belt further includes a fastener secured to the distal ends of the second adjustable belt.

10. The therapeutic support garment of claim 5, wherein the second adjustable belt further comprises a second releasable fastener for fastening the second adjustable belt below the first adjustable belt.

11. The therapeutic support garment of claim 10, wherein the second adjustable belt is a continuous band of elastic material with opposed overlapping ends thereof carrying the second releasable fastener.

12. A therapeutic support garment comprising:
a panty layer having a support channel wherein the support channel is stitched above the panty layer;
an adjustable belt supported by the support channel;
a releasable fastener for fastening the adjustable belt around a lower section of a user's abdomen; and
a crotch portion having a cushioning layer for protecting an incision site.

13. The therapeutic support garment of claim 12, wherein the adjustable belt is approximately three inches in width.

14. The therapeutic support garment of claim 12, wherein the adjustable belt is a continuous band of elastic material with opposed overlapping ends thereof carrying the releasable fastener.

15. A therapeutic support garment comprising:
a panty layer having a first stitched channel and a second stitched channel;
the first stitched channel for supporting a first adjustable belt;
the second stitched channel for supporting a second adjustable belt;
the first adjustable belt having a releasable fastener for fastening the adjustable belt around a lower section of a user's abdomen; and
a crotch portion having a stabilizing cushion for protecting an incision site.

16. The therapeutic support garment of claim 15, wherein the first adjustable belt and the second adjustable belt are approximately three inches in width.

17. The therapeutic support garment of claim 15, wherein the first adjustable belt is a continuous band of elastic material with opposed overlapping ends thereof carrying the releasable fastener.

18. The therapeutic support garment of claim 15, wherein the second adjustable belt further comprises a second releasable fastener for fastening the second adjustable belt, the second adjustable belt being below the first adjustable belt.

19. The therapeutic support garment of claim 18, wherein the second adjustable belt is a continuous band of elastic material with opposed overlapping ends thereof carrying the second releasable fastener.
